(19) United States
(12) Patent Application Publication
(10) Pub. No.: US 2005/0043289 A1
(43) Pub. Date: Feb. 24, 2005

(54) Crystalline hydrochloride of 7-(((5-amino-1,2,4-thiadiazol-3-yl) (fluoromethoxyimino)acetyl)amino)-3-((imino-1-piperazinylmethyl)methylhydrazono)-methyl-3-cephem-4-carboxylic acid

(76) Inventors: Julia Greil, Kramsach (AT); Johannes Ludescher, Breitenbach (AT); Siegfried Wolf, Brixlegg (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/499,616

(22) PCT Filed: Jan. 31, 2003
(86) PCT No.: PCT/EP03/00971

(30) Foreign Application Priority Data
Feb. 1, 2002 (AT) 171/2002

(51) Int. Cl. A61K 31/545; C07D 501/14

(52) U.S. Cl. 514/202; 540/222

(57) ABSTRACT

A crystalline hydrochloride of the compound 7-{[5-amino-1,2,4-thiadiazol-3-yl-(fluoromethoxyimino)acetyl)amino)-3-((imino-1-piperazinylmethyly)methylhydrazono]-methyl-3-cephem-4carboxylic acid and the pharmaceutical use thereof.

The present invention relates to cephalosporins, such as the compound 7-{[(5-amino-1,2,4-thiadiazol-3-yl)(fluoromethoxy-imino)acetyl]amino}-3-[(imino-1-piperazinylmethyl)-methylhydrazono]-methyl-3-cephem-4-carboxylic acid, e.g. the compound 7-{[(5-amino-1,2,4-thiadiazol-3-yl)-(Z)-fluoromethoxy-imino)acetyl]amino}-3(E)-[(imino-1-piperazinylmethyl)-methylhydrazono]-methyl-3-cephem-4-carboxylic acid of formula

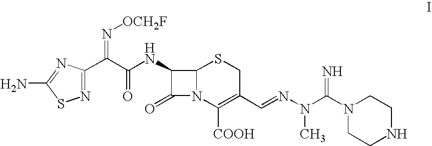

From e.g. W098/43981 it is known that a compound of formula I has pharmaceutical activity and may be used e.g. as an antimicrobial agent against diseases which may be caused by microbes, e.g. bacteria, e.g. for the treatment of diseases associated with bacterial infections. According to example 1 of W098/43981 a compound of formula I may be obtained in the form of a lyophilised monohydrochloride, e.g. by
precipitation of a compound of formula I in the form of a trihydrochloride,
conversion into the form of a monohydrochloride by using a chromatographic method, and
lyophilisation.

We now surprisingly have found that a compound of formula I may be obtained in the form of a crystalline salt. A compound of formula I in the form of a crystalline salt is new.

In one aspect the present invention provides a compound of formula I in the form of a crystalline salt.

A compound of formula I may be obtained in the form of a hydrochloride in crystalline form, e.g. including a monohydrochloride and a dihydrochloride of a compound of formula I. A compound of formula I in the form of a crystalline hydrochloride is hereinafter designated as “a compound of (according to) the present invention” or “a (mono- or di-)hydrochloride(s) of (according to) the present invention”.

In another aspect, the present invention provides a compound of formula I in the form of a crystalline hydrochloride, e.g. a crystalline monohydrochloride or a crystalline dihydrochloride.

We have found that a compound of the present invention may exist in the form of a solvate, for example a hydrate.

In another aspect, the present invention provides
a compound of formula I in the form of a crystalline hydrochloride in the form of a solvate, e.g. a hydrate, and
a compond of formula I in the form of a monohydrochloride in the form of a solvate, e.g. a trihydrate.

A compound of the present invention may be prepared from aqueous solution containing HCl, optionally after inoculation. Said aqueous solution contains a compound of formula I, HCl and beside water as a solvent optionally an organic solvent may be present. Optionally, an anti-solvent is added. An anti-solvent as used herein is meant to be a solvent in which a compound of the present invention has poorer solubility than in water. Preferably an alcohol, such as ethanol or isopropanol, or a ketone, such as acetone is used as an anti-solvent. More preferably the anti-solvent is an alcohol. The weight ratio of water and anti-solvent is not critical. If an alcohol is used, a weight ratio of water to alcohol of 4:1 to 100:75 has shown to be advantageous. Optionally, further anti-solvent, e.g. an alcohol may be added, e.g. in order to increase yields. An aqueous solution of a compound of formula I containing HCl contains at least 1 equivalent of HCl (to produce a monohydrochloride of the present invention) or at least 2 equivalents of HCl (to produce a dihydrochloride of the present invention), preferably more, e.g. 2 to 10 (monohydrochloride) or 3 to 10 (dihydrochloride), such as 2 to 6 (monohydrochloride) or 3 to 6 (dihydrochloride) equivalents of HCl, based on a compound of formula I.

It is one advantage of the present invention that a compound of the present invention may be obtained directly, that means, without isolating a compound of formula I, from a preparation process for the production of a compound of formula I.

A compound of formula I may be prepared according, e.g. analogously, to a method as conventional, e.g. as described in the prior art. Preferably, a compound of formula I is prepared in accordance with the following reaction SCHEME 1:

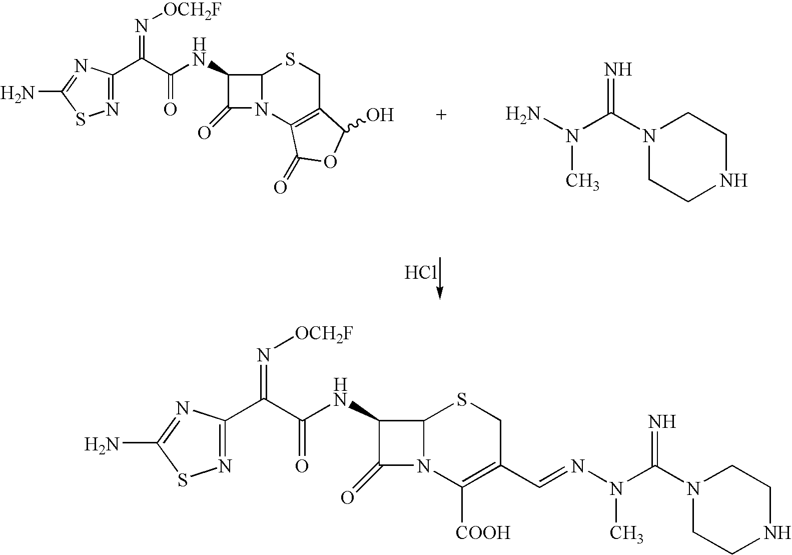

The reaction according to SCHEME 1 is carried out in (a mixture of) organic reaction solvent and in the presence of aqueous HCl. A solution of a compound of formula I in the form of a hydrochloride in a mixture of organic reaction solvent and water may be obtained. To obtain a compound of the present invention, the main quantity of organic reaction solvent is removed from said solution, for example by evaporation or extraction. An aqueous solution is obtained containing HCl and containing as a solvent primarily water together with residual amounts of organic reaction solvent which aqueous solution is treated optionally with water and/or optionally with an anti-solvent, in order to improve yields. A compound of the present invention may crystallise, optionally after inoculation. Inoculation crystals may be obtained e.g. in pre-trials.

In a further aspect, the present invention provides a process for the production of a crystalline hydrochloride of a compound of formula I, comprising crystallising a hydrochloride of a compound of formula I, from water, a mixture of water and alcohol a mixture of water and ketone, or a mixture of water and alcohol and ketone, in the presence of hydrochloric acid.

In one embodiment of the present invention, a monohydrochloride of the present invention may be prepared by adding HCl to an aqueous solution of a compound of formula I followed by adjustment of a pH value of 3 to 5.5 by addition of a suitable base. Suitable bases are, for example, organic bases, e.g. alkylamines, such as (C₁₋₆)mono-, di- and trialkylamines, preferably (C₁₋₆)trialkylamines, e.g. tributylamine, or inorganic bases, such as carbonates or bicarbonates, e.g. Na₂CO₃ or NaHCO₃.

In another embodiment of the present invention, a dihydrochloride of the present invention may be prepared by adding HCl in an appropriate amount to an aqueous solution of a compound of formula I, e.g. to adjust a pH which is lower than 3, e.g. 1 and below, such as from pH-1 to pH 3, e.g. from pH-1 to pH 1; the addition of a base is not necessary.

In a preferred embodiment of the present invention a dihydrochloride of the present invention is isolated in a step following SCHEME 1 by removing the reaction solvent and by adding water and optionally an anti-solvent. A dihydrochloride of the present invention as obtained may be isolated and converted into a monohydrochloride of the present invention in aqueous or aqueous/organic solution, e.g. in water or in a combination of water with a ketone, e.g. acetone, or with an alcohol, e.g. isopropanol, by adjusting the pH value as described above.

We have found that a compound of the present invention, e.g. a monohydrochloride of the present invention, may be obtained in non-solvatized form or in the form of a solvate, e.g. a hydrate. A monohydrochloride, produced according to the present invention, may be obtained in the form of a trihydrate, which, after appropriate drying, has a water content of ca. 7% to 10%, such as 7.3% to 9.6% (theory: 8.2%). The monohydrochloride in the form of a trihydrate may be dried, e.g. over P₂O₅, to a water content of 1.5%. In the presence of environmental moisture, e.g. in the stress test, said trihydrate may absorb water, e.g. up to a quantity corresponding to a pentahydrate (ca. 12.9% to 15.0%). The chloride content of the monohydrochloride in the form of a trihydrate is between 5% and 6% (theory: 5.5%).

Although the monohydrochloride of the present invention in the form of a trihydrate may be sensitive to moisture, it has shown to be an appropriate form for further applications, e.g. pharmaceutical administration.

It has also been found that a dihydrochloride according to the present invention, which is e.g. obtained by a process according to the present invention, may be present in the form of a solvate. A dihydrochloride of the present invention, e.g. prepared as indicated in the examples may contain a residual amount of water, e.g. from 3% to 15% (w/w), preferably from 3% to 12% (w/w). If crystallisation is carried out in an alcohol, e.g. ethanol, containing solution, about 0.05 to 3% (w/w) alcohol may be present, e.g. about 3% (w/w) ethanol, in a dihydrochloride of the present invention.

The crystalline structure of a mono- and of a dihydrochloride according to the present invention can be determined by powder X-ray diffraction patterns.

A hydrochloride according to the present invention may be obtained in a high degree of purity. A crystalline monohydrochloride of a compound of formula I in the form of a trihydrate may be obtained in substantially pure form, e.g. more than 99.9% purity, such as about 100% purity (correspondintg to 85.8% based on the free base of a compound of formula I). A high purification effect is obtained in a preparation process of a compound of formula I by crystallisation of a hydrochloride according to the present invention, which may be surprisingly achieved under conditions as described above. Complex purification methods, e.g. chromatographic methods, may thus be avoided.

Furthermore, it has been found that a compound of formula I in the form of a crystalline hydrochloride, both in solid form and in solution, may have high stability, e.g. as necessary for administration. E.g. we have found that a monohydrochloride of the present invention in solid form may be stored for at least 4 weeks, e.g. at 5° C., with practically no or only slight decomposition of a compound of formula I.

We have also found that a monohydrochloride of the present invention also remains stable for at least one week in an aqueous solution at pH values of 3-5 at 5° C.

Furthermore, we have found that a monohydrochloride of the present invention shows appropriate solubility in an aqueous medium for pharmaceutical administration. E.g. a compound of the present invention may have a solubility of ≧2% (w/v) in water at 5°C., and of about 10% (w/v) at room temperature. The pharmaceutical activity of a compound of formula I in free base form, for example the antibiotic activity as described in WO98/43981, is comparable to the pharmaceutical activity of a compound of the present invention. Thus, a compound of the present invention is appropriate for pharmaceutical administration.

In another aspect the present invention provides the use of a compound of the present invention as a pharmaceutical, e.g. as an antibiotic.

In a further aspect the present invention provides a process for the production of a medicament for treating antimicrobial, e.g. antibacterial, infections, which is characterised in that a compound of the present invention is used to produce said medicament.

The compounds of the present invention exhibit pharmaceutical activity and surprising low toxicity and are therefore useful as pharmaceuticals. In particular, the compounds of the present invention show antimicrobial, e.g. antibacterial activity against aerobic and anaerobic growing bacteria, e.g. gram negative and gram positive bacteria such as Enterobacter, e.g. Enterobacter cloacae; Enterococcus, e.g. Enterococcus faecalis; Moraxella, e.g. Moraxella catarrhalis; Haemophilus, e.g. Haemophilus influenza; Klebsiella, e.g. Klebsiella edwardsii, Klebsiella pneumoniae; Streptococcus, e.g. Streptococcus pyogenes; Staphylococcus, e.g. Staphylococcus aureus MSSA (methicillin sensitive strains); Staphylococcus aureus MRSA (methicillin resistant strains); Escherichia coli; Proteus, e.g. Proteus mirabilis; Salmonella, e.g. Salmonella typhimurium; Serratia, e.g. Serratia marcescens; Pneumococci, e.g. Streptococcus pneumoniae (penicillin resistant and multi-drug resistant strains); in vitro in the Agar Dilution Test and/or Micro Dilution Test for bacteria according to National Committee for Clinical Laboratory Standards (NCCLS) 1993,
Document M7-A3, Vol. 13, No.25: “Methods for dilution Antimicrobial Susceptibility Tests for Bacteria that Grow Aerobically”—Third Edition, Approved Standard”; and
Document M11 -A3 for anaerobic bacteria in a concentration from about 0.001 to about 50 μm/ml (MIC), e.g. using strains including Staphylococcus aureus (ATCC 29213 and ATCC 9144); Enterococcus faecalis (ATCC 29212); Haemophilus influenza (NTCC 49247 and NCTC 11931); Escherichia coli (ATCC 25922 and ATCC 35218); Klebsiella pneumoniae (NCTC 11228); Klebsiella edwardsii (NCTC 10896); and in vivo in the septicaemia mouse model, in accordance to the method description Nr. 159 A-5, approved by Austrian Health Authorities (MA 58, no. 2968/95 of 12 Oct.1995), e.g. when administered at dosages from about 0.05 to 50 mg/kg body weight, such as 0.1 to 50 mg/kg body weight (ED₅₀ values). E.g., mice are infected with an ED 95% of Staphylococcus aureus (ATCC 4995), Streptococcus pyogenes (ATCC 29218), Escherichia coli (Δ 12 NFI culture collection) and are treated 1.5 and 24 hours after infection. The ED 50% values ranging form ca. 0.2 to 50 mg/kg body weight are calculated by Probit analysis of the administered dosages of compounds. Activity is determined by numbers of surviving animals per group of 8 mice per dosage unit day 5 after infection. The compounds of the invention show an surprising overall activity spectrum.

It has, for example, been determined that the MIC (μg/ml) of the compound of Example 2 or 4 against, for example Enterococcus faecalis is of about 0.1 to 0.8; against Staphylococcus aureus (MSSA) is of about 0.2 to 0.8; against methicillin resistant Staphylococcus aureus is of 0.8 to 12.6; against multi-drug resistant Pneumococcus is of 0.4 to 0.8.

The compounds of the present invention are therefore useful in the treatment of microbial, e.g. bacterial diseases, e.g. the treatment of diseases associated with bacterial infections. Treatment includes treatment and prevention in humans and animals.

For this indication, the appropriate dosage will, of course, vary depending upon, for example, the compound of the present invention employed, the host, the mode of administration and the nature and severity of the conditions being treated. However, in general, for satisfactory results in larger mammals, for example humans, an indicated daily dosage is in the range from about 0.05 to 5 g, for example 0.1 to about 2.5 g of a compound of the present invention conveniently administered, for example, in divided doses up to four times a day.

A compound of the invention may be administered by any conventional route, for example parenterally in the form of injectable solutions or suspensions, e.g. in analogous manner to cefotaxime, or orally, e.g. in the form of tablets or capsules.

The crystalline monohydrochloride of a compound of formula I, preferably in the form of its trihydrate, is the preferred compound of the invention for use as an antimicrobial, e.g. antibacterial, agent.

It has for example been determined that the MIC (μg/ml) of the compound of example 2 or 4 against, for example Enterobacter cloacae is about 0.025 to 12.8 and, for example cefotaxime shows an MIC (μg/ml) of about 0.125 to >256.

It is therefore indicated that for the treatment of microbial diseases, e.g. bacterial diseases, such as diseases associated with bacterial infections, the preferred compounds of the invention may be administered to larger mammals, for example humans, by similar modes of administration at similar dosages than conventionally employed with cefotaxime.

In another aspect the present invention provides a pharmaceutical composition comprising a compound of the present invention in association with at least one pharmaceutical excipient, e.g. carrier or diluent.

Such compositions may be manufactured in conventional manner. Unit dosage form may contain, for example 100 mg to about 2 g, for example 250 mg to about 1 g, for example 250 mg to about 500 mg, such as to about 500 mg.

As medicaments, the active ingredient, i.e. a compound of the present invention, may be administered alone or in suitable medicinal forms (pharmaceutical compositions) together with one or more inorganic or organic, pharmaceutically acceptable excipient. Pharmaceutically acceptable excipient includes carrier(s) and diluent(s). For example, a compound of the present invention may be used in injection or instillation preparations, which contain a quantity of a compound of the present invention that is sufficient to attain an optimum blood level, that is, about 100 mg to 500 mg per application. For this application, the dosage to be administered depends on the compound used and the type of administration, as well as the type of treatment. With larger mammals satisfactory results may be obtained when administering a daily dose of about 0.5 to 6 g.

In another aspect the present invention provides the use of a compound of the present invention or the use of a composition comprising a compound of the present invention in association with at least one pharmaceutical excipient as a pharmaceutical.

In a further aspect the present invention provides
a method of treatment of microbial diseases, such as treatment of diseases associated with bacterial infections e.g. caused by bacteria selected from Enterobacter, Enterococcus, Moraxella, Haemophilus; Klebsiella, Streptococcus, Staphylococcus, Escherichia, Proteus, Salmonella, Serratia or Pneumococci, which comprises administering to a subject in need of such treatment, e.g. a human or an animal, such as an animal, an effective amount of a compound of the present invention, e.g. in the form of a pharmaceutical composition according to the present invention; and a compound of of the present invention for use in the preparation of a medicament for the treatment of microbial diseases, e.g. the treatment of diseases associated with bacterial infections, for example of diseases caused by bacteria selected from Enterobacter, Enterococcus, Moraxella, Haemophilus; Klebsiella, Streptococcus, Staphylococcus, Escherichia, Proteus, Salmonella, Serratia or Pneumococcus.

In the following examples, all temperatures are given in ° Celsius. The following abbreviations are used:
AcCN: acetonitrile
DIMAC: N,N-dimethylacetamide
EtOAc: ethyl acetate
MIC: minimum inhibitory concentration

### EXAMPLE 1

### 7-{[(5-amino-1,2,4-thiadiazol-3-yl)-(Z)-fluoromethoxyimino)acetyl]amino}-3-[(imino-1-piperazinylmethyl)methylhydrazono]-methyl-3-cephem-4-carboxylic acid

a) N-(1,4,5a,6-tetrahydro-3-hydroxy-1,7-dioxo-3H,7H-aceto(2,1-b)furo(3,4-d)(1,3)-thiazin-6-yl)-2-(5-amino-1,2,4-thiadiazol-3-yl)-(Z)-2-(fluoromethoxyimino)-acetic acid amid

(Hydroxylactone of 7-{[(5-amino-1,2,4-thiadiazol-3-yl)-(Z)-(fluoromethoxyimino)acetyl]amino}-3-formyl-3-cephem-4-carboxylic acid)

A suspension of 10 g of 7-amino-3-formyl-3-cephem-4-carboxylic acid in a mixture of 220 ml of CH₂Cl₂ and 80 ml of AcCN is stirred at 0° with 43 ml of N,O-bis(trimethylsilyl)-acetamide. 15.7 g of (5-amino-1,2,4-thiadiazol-3-yl)-(Z)-2-fluoromethoxyimino-acetic acid chloride are added to a solution obtained. A mixture obtained is stirred at ca. 0° for about one hour, diluted with 1250 ml of AcCN containing 70 ml of H₂O, 12% aqueous ammonia is added and a pH value of 3.5 is adjusted. A mixture obtained is diluted with 2.5 l of H₂O and extracted with EtOAc. An organic phase obtained is dried and concentrated and a concentrate obtained is stirred for ca. 1 hour at ca. 20° with 100 ml of AcCN. Crystalline N-(1,4,5a,6-tetrahydro-3-hydroxy-1,7-dioxo-3H,7H-aceto(2,1-b)furo(3,4-d)(1,3)-thiazin-6-yl)-2-(5-amino-1,2,4-thiadiazol-3-yl)-(Z)-2-(fluoromethoxyimino)-acetic acid amid precipitates from the resulting mixture, is filtered off and dried.

¹H-NMR: 3.65 (m, 2×AB quartet, 2H, SCH₂); 5.18 (d, J=5 Hz, 1H, β-lactam-H); 5.83 (d, J=55 Hz, 2H, CH₂F); 6.03 (dd, J=5 and 8.3 Hz, 1H, β-lactam-H); 6.24 and 6.30 (s, 1H, O—CH—O); 8.25 (broad singulet, 2H, NH₂); 9.89 and 9.87 (d, J=8,3 Hz, 1H, NH).

b) 7-{[(5-amino-1,2,4-thiadiazol-3-yl)-(Z)-(fluoromethoxyimino)acetlyl]amino}-3(E)-[(imino-1-piperazinylmethyl)methylhydrazonol-methyl-3-cephem-4-carboxylic acid

A solution of 132.7 g of 1-(1-methylhydrazino)iminomethyl)piperazine in the form of a hydrochloride in a mixture of 300 ml of 2N aqueous HCl and 516 ml of DIMAC is mixed at ca. 3° with 154 g of N-(1,4,5a,6-tetrahydro-3-hydroxy-1,7-dioxo-3H,7H-aceto(2,1-b)furo(3,4-d)-(1,3)-thiazin-6-yl)-2-(5-amino-1,2,4-thiadiazol-3-yl)-(Z)-2-fluoromethoxyimino)acetic acid amide, a mixture obtained is stirred for ca. 1 day, 300 ml of H₂O are added and DIMAC is removed from the mixture obtained. An aqueous mixture obtained contains 7-{[(5-amino-1,2,4-thiadi-azol-3-yl)-(Z)-fluoromethoxyimino)acetyl]amino}-3(E)-[(imino-1-piperazinylmethyl) methyl-hydrazono]-methyl-3-cephem-4-carboxylic acid in the form of a hydrochloride.

### EXAMPLE 2

### 7-{[(5-amino-1,2,4-thiadiazol-3-yl)-(Z)-(fluoromethoxyimino)acetyl]amino}-3-[(imino-1-piperazinylmethyl)methylhydrazono]-methyl-3-cephem-4-carboxylic acid in the form of a monohydrochloride and in the form of a trihydrate

1000 ml of water, 180 ml of isopropanol and 195 ml of tributylamine, dissolved in 600 ml of isopropanol, are added under vigorous stirring and cooling (pH value from 3 to 4) to a cooled aqueous mixture obtained according to example 1, further 225 ml of isopropanol are added. 7-{[(5-amino-1,2,4-thiadiazol-3-yl)-(Z)-fluormethoxyimino)acetyl]amino}-3-[(imino-1-piperazinylmethyl)methyl-hydrazono]-methyl-3-cephem-4-carboxylic acid in the form of a monohydrochloride and in the form of a trihydrate crystallizes from a mixture obtained, optionally after inoculation, whilst cooling, is filtrated off, washed and dried.

Water content (Karl Fischer): 9.6%. Water content constant (±6%) over 6 weeks. HCl (titration): 5.5%. Isopropanol-content: 0.05%. Content of a compound of formula I (based on the free base): 84.%. pH and [β]²⁰_{D} of a 1% solution in water: pH: 4.3; [β]²⁰₅₈₉=−167±1°

### EXAMPLE 3

### 7-{[(5-amino-1,2,4-thiadiazol-3-yl)-(Z)-(fluoromethoxyimino)acetyl]amino}-3-[(imino-1-piperazinylmethyl)methylhydrazono]-methyl-3-cephem-4-carboxylic acid in the form of a dihydrochloride

600 ml of cooled ethanol are added under stirring to a cooled aqueous mixture obtained according to example 1. 7-{[(5-amino-1,2,4-thiadiazol-3-yl)-(Z)-(fluormethoxyimino)acetyl]-amino}-3-[(imino-1-piperazinylmethyl)methylhydrazono]-methyl-3-cephem-4-carboxylic acid crystallizes in the form of a dihydrochloride, optionally after inoculation, is filtrated off, washed and dried. Water content: 4.5%. HCl (titration): 10.5%. Ethanol-content: 3%. Content of a compound of formula I (based on the free base): 76.2%.

### EXAMPLE 4

7-{[(5-amino-1,2,4-thiadiazol-3-yl)-(Z)-(fluoromethoxyimino)acetyl]amino}-3-[(imino-1-piperazinylmethyl)methylhydrazono]-methyl-3-cephem-4-carboxylic acid in the form of a monohydrochloride and in the form of a trihydrate

11 ml of a saturated NaHCO₃ solution and 50 ml of isopropanol are added to a cooled solution of 10 g of 7-{[(5-amino-1,2,4-thiadiazol-3-yl)-(Z)-(fluormethoxyimino)-acetyl]-amino}-3-[(imino-1-piperazinylmethyl)methylhydrazono]-methyl-3-cephem-4-carboxylic acid in the form of a dihydrochloride in 90 ml of H₂O. A pH value of 3 to 4 is adjusted. 7-{[(5-amino-1,2,4-thiadiazol-3-yl)-(Z)-fluormethoxyimino)acetyl]amino}-3-[(imino-1-piperazinylmethyl)methyl-hydrazono]-methyl-3-cephem-4-carboxylic acid in the form of a monohydrochloride and in the form of a trihydrate crystallizes, optionally after inoculation, is filtrated off, washed and dried. Water content: 9.3%. HCl (titration): 5.4%. Isopropanol: 0.05%. Content of a compound of formula I (based on the free base): 84.1%.

### EXAMPLE 5

### Conversion of 7-{[(5-amino-1,2,4-thiadiazol-3-yl)-(Z)-(fluoromethoxyimino)acetyl]amino}-3-{(imino-1-piperazinylmethyl)methylhydrazono]-methyl-3-cephem-4-carboxylic acid in the form of a dihydrochloride into the form of a monohydrochloride

10 g of 7-{[(5-amino-1,2,4-thiadiazol-3-yl)-(Z)-(fluoromethoxyimino)acetyl]amino}-3-{(imino-1-piperazinylmethyl)methylhydrazono]-methyl-3-cephem-4-carboxylic acid in the form of a dihydrochloride are dissolved in 90 ml of cooled water. The pH of the solution obtained is adjusted to a value of 3.0 to 4.0 and isopropanol is added under stirring. Stirring is continued overnight at a temperature below 5°. 7-{[(5-amino-1,2,4-thiadiazol-3-yl)-(Z)-(fluoromethoxy-imino)acetyl]amino}-3-{(imino-1-piperazinylmethyl)methylhydrazono]-methyl-3-cephem-4-carboxylic acid in the form of a monohydrochloride in crystalline form is obtained.

The crystalline structure of the compounds obtained according to examples 2 to 5 is confirmed by their X-Ray powder diffraction pattern. The X-Ray powder diffraction patterns are determined by use of a
X-Ray Powder Diffractometer D-8 (AXS-BRUKER) theta-theta goniometer, sample changer
target: Copper, Kα1+Kα2λ=1.50406 Angstroem
parallel beam optics (receiving soller-slit: 0.07 mm)
Scinitillation counter, standard sample holder.

In Tables 1, 1a, 2 and 2a below “d” denotes the interplanar spacing and “I/Iₒ” denotes the relative intensity.

Table 1 and Table 1a below show “d” and “I/Iₒ” for a crystalline dihydrochloride obtained according to Example 3 (Table 1a is more detailed than Table 1):

TABLE 1

| d(Å) | I/I₀ |
|---|---|
| 21.02 | 100 |
| 9.392 | 27 |
| 3.572 | 37 |
| 3.243 | 21 |

TABLE 1a

| d(Å) | I/I₀ |
|---|---|
| 21.02 | 100 |
| 10.94 | 8 |
| 10.48 | 8 |
| 10.03 | 12 |
| 9.392 | 27 |
| 7.883 | 6 |
| 7.274 | 9 |
| 6.846 | 3 |
| 6.090 | 9 |
| 6.025 | 11 |
| 5.673 | 5 |
| 5.456 | 7 |
| 5.379 | 6 |
| 5.172 | 6 |
| 5.005 | 6 |
| 4.879 | 2 |
| 4.574 | 19 |
| 4.468 | 10 |
| 4.367 | 8 |
| 4.181 | 6 |
| 4.056 | 4 |
| 3.914 | 6 |
| 3.627 | 13 |
| 3.572 | 37 |
| 3.537 | 19 |
| 3.451 | 9 |
| 3.404 | 10 |
| 3.337 | 15 |
| 3.243 | 21 |
| 3.046 | 6 |
| 3.004 | 5 |
| 2.856 | 7 |
| 2.822 | 10 |
| 2.779 | 4 |
| 2.736 | 4 |
| 2.666 | 6 |
| 2.640 | 6 |
| 2.611 | 10 |
| 2.535 | 9 |
| 2.501 | 5 |
| 2.433 | 5 |
| 2.388 | 3 |
| 2.339 | 2 |
| 2.308 | 2 |

Table 2 and Table 2a show “d” and “I/Iₒ” for a crystalline monohydrochloride obtained according to Examples 2or 4 (Table 2a is more detailed than Table 2):

TABLE 2

| d(Å) | I/I₀ |
|---|---|
| 10.18 | 68 |
| 6.257 | 100 |
| 4.961 | 61 |
| 4.110 | 68 |
| 3.594 | 56 |
| 3.576 | 63 |

TABLE 2a

| d(Å) | I/I₀ |
|---|---|
| 12.75 | 21 |
| 10.18 | 68 |
| 8.253 | 40 |
| 6.990 | 26 |
| 6.845 | 11 |
| 6.599 | 11 |
| 6.257 | 100 |
| 5.875 | 30 |
| 5.495 | 16 |
| 5.418 | 23 |
| 5.087 | 26 |
| 4.961 | 61 |
| 4.757 | 21 |
| 4.592 | 6 |
| 4.370 | 7 |
| 4.193 | 13 |
| 4.110 | 68 |
| 3.944 | 9 |
| 3.645 | 12 |
| 3.594 | 56 |
| 3.576 | 63 |
| 3.509 | 23 |
| 3.489 | 19 |
| 3.457 | 21 |
| 3.422 | 20 |
| 3.382 | 43 |
| 3.311 | 22 |
| 3.297 | 21 |
| 3.181 | 28 |
| 3.128 | 48 |
| 3.076 | 25 |
| 3.065 | 20 |
| 3.005 | 15 |
| 2.933 | 22 |
| 2.920 | 26 |
| 2.847 | 2 |
| 2.774 | 5 |
| 2.737 | 10 |
| 2.705 | 18 |
| 2.697 | 17 |
| 2.638 | 4 |
| 2.589 | 9 |
| 2.584 | 9 |
| 2.574 | 6 |
| 2.544 | 27 |
| 2.503 | 9 |
| 2.465 | 5 |
| 2.429 | 4 |
| 2.394 | 5 |
| 2.370 | 9 |
| 2.361 | 9 |
| 2.329 | 10 |

1. A compound of formula 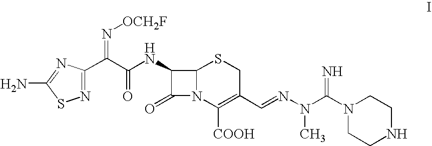
in the form of a crystalline salt.

2. A compound according to claim 1 in the form of a crystalline hydrochloride.

3. A compound according to claim 1 in the form of a crystalline monohydrochloride.

4. A compound according to claim 1 in the form of a crystalline dihydrochloride.

5. A compound according to claim 1 in the form of a solvate.

6. A compound according to claim 1 in the form of a monohydrochloride and a trihydrate.

7. A pharmaceutical composition comprising a compound of claim 1 in association with at least one pharmaceutically acceptable excipient.

8. A pharmaceutical composition according to claim 7 comprising a compound of formula I in the form of a monohydrochloride and a trihydrate.

9. (cancelled).

10. A method of treatment of diseases associated with bacterial infections comprising administering to a subject in need of such treatment an effective amount of a compound of claim 1.
